(19) United States
(12) Patent Application Publication
(10) Pub. No.: US 2012/0123157 A1
(43) Pub. Date: May 17, 2012

(54) FORMIC ACID MANUFACTURING SYSTEM AND METHOD OF MANUFACTURING FORMIC ACID

(75) Inventors: Hsiao-Ping HUANG, Taipei City (TW); Ming-Jer Lee, Taipei City (TW); Hao-Yen Lee, Taipei City (TW); Jyun-Hong Chen, Taipei City (TW)

(73) Assignee: National Taiwan University, Taipei City (TW)

(21) Appl. No.: 12/944,240

(22) Filed: Nov. 11, 2010

(51) Int. Cl. C07C 51/12; B01J 19/00

(52) U.S. Cl. 562/519; 422/187

(57) ABSTRACT

Formic acid manufacturing system and method of manufacturing formic acid. The formic acid manufacturing system includes a reactor, a first separation device, a second separation device, a reactive distillation device and a third separation device. Methyl formate is produced in the reactor by processing a conbonylation reaction of external carbon monoxide and methanol. Water is added into the reactive distillation device externally, and formic acid and methanol is produced in the reactive distillation device by hydrolyzing methyl formate with water. A formic acid solution is obtained from an output of the third separation device.

### BACKGROUND OF THE INVENTION

1. Field of the Invention

The invention relates in general to formic acid manufacturing system and method of manufacturing formic acid, and more particularly to formic acid manufacturing system and method of manufacturing formic acid which can produce high purity formic acid.

2. Description of the Related Art

Formic acid is an organic chemical raw material. In the leather industry, formic acid is a substitute for inorganic acid synthesis. Formic acid can be used to de-ash for tanning, neutralize lime and prevent the wet leather from moldy. Formic acid also can inhibit the growth of mold and be applied to forage and grain preservation. Formic acid also can be the acid coagulant of emulsion and the coagulant of cement etc.

Because formic acid and water may form azeotropic composition, it is not easy to produce high purity formic acid. Otherwise, it is necessary to utilize a lot of units to break the azeotropic composition to get high purity formic acid. High purity formic acid has higher economic value, but the cost of producing the high purity formic acid is very expansive. Therefore, it is a subject of the industrial endeavors to research a system that can produce high purity formic acid and save more cost at the same time.

### SUMMARY OF THE INVENTION

The invention is directed to formic acid manufacturing system and method of manufacturing formic acid which provide a system and a method to produce high purity formic acid.

According to a first aspect of the present invention, a formic acid manufacturing system is provided. The formic acid manufacturing system comprises a reactor, a first separation device, a second separation device, a reactive distillation device and a third separation device. An external carbon monoxide reacts with a methanol to produce a first mixture in the reactor. A first separation device is used for receiving the first mixture. The first separation device separates the first mixture into a second mixture and a third mixture. The second mixture is conveyed to the reactor. A second separation device is used for receiving the third mixture. The second separation device separates the third mixture into a fourth mixture and a fifth mixture. The fourth mixture is conveyed to the reactor. A reactive distillation device is used for receiving the fifth mixture and an external water. The fifth mixture reacts with the external water to produce a sixth mixture in the reactive distillation device. A third separation device is used for receiving the sixth mixture. The third separation device separates the sixth mixture into a seventh mixture and a eighth mixture. The seventh mixture is conveyed to the reactor. The eighth mixture is formic acid solution.

According to a second aspect of the present invention, a method of manufacturing formic acid is provided. The formic acid manufacturing system comprises the following steps. Convey an external carbon monoxide and a methanol to a reactor. The external carbon monoxide reacts with the methanol to produce a first mixture in the reactor; Convey the first mixture to a first separation device to separate into a second mixture and a third mixture. The second mixture is conveyed to the reactor. Convey the third mixture to a second separation device to separate into a fourth mixture and a fifth mixture. The fourth mixture is conveyed to the reactor. Convey the fifth mixture and an external water to a reactive distillation device. The fifth mixture reacts with the external water to produce a sixth mixture. Convey the sixth mixture to a third separation device to separate into a seventh mixture and a eighth mixture. The seventh mixture is conveyed to the reactor. The eighth mixture is formic acid solution.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is schematic diagram showing formic acid manufacturing system according to a first embodiment of the invention.

FIG. 2 shows a flow chart showing the method of manufacturing formic acid according to a first embodiment of the invention.

FIG. 3 is schematic diagram showing formic acid manufacturing system according to a second embodiment of the invention.

FIG. 4 shows a flow chart showing the method of manufacturing formic acid according to a second embodiment of the invention.

### DETAILED DESCRIPTION OF THE INVENTION

The carbon monoxide and the methanol process a conbonylation reaction to produce the methyl formate as shown in chemical formula (I). The water and the methyl formate process a hydrolysis reaction to produce the formic acid and the methanol as shown in chemical formula (II). The net reaction of chemical formula (I) and chemical formula (II) is carbon monoxide reacting with water to produce formic acid as shown in chemical formula (III). As mentioned above, the production methyl formate of chemical formula (I) can be the reactant of chemical formula (II) and the production methanol of chemical formula (II) can be the reactant of chemical formula (I) to prevent the waste of raw materials. The components of chemical formula (I) and (II) are ranked in order of boiling point (B. P.) from low to high in Table 1. The boiling point of methyl formate, methanol, water and formic acid are 31.79° C., 64.53° C., 100.02° C. and 100.77° C. respectively. Particularly, an azeotropic composition is formed by formic acid and water. The mole fraction of formic acid in the azeotropic composition is 0.5821 and the azeotrpic temperature of the azeotropic composition is 107.73° C. at 1 atm. Utilize the different boiling point of each component can separate these components. For example, by using distillation column the high boiling point material can be withdrawn from the bottom of the distillation column while the low boiling point material can be withdrawn from the top of the distillation column. Thus, the following embodiments can get high purity formic acid. The following have two embodiments to illustrate.

CH3OH+CO [symbol] HCOOCH3 (I)

HCOOCH3+H2O [symbol] HCOOH+CH3OH (II)

H2O+CO [symbol] HCOOH (III)

TABLE 1

| Component | B.P. (° C.) (at 1 atm) | Mole fraction |
|---|---|---|
| Methyl formate | 31.79 | — |
| Methanol | 64.53 | — |
| Water | 100.02 | — |
| Formic acid | 100.77 | — |
| Formic acid/water | 107.73 | 0.5821/0.4179 |

### First Embodiment

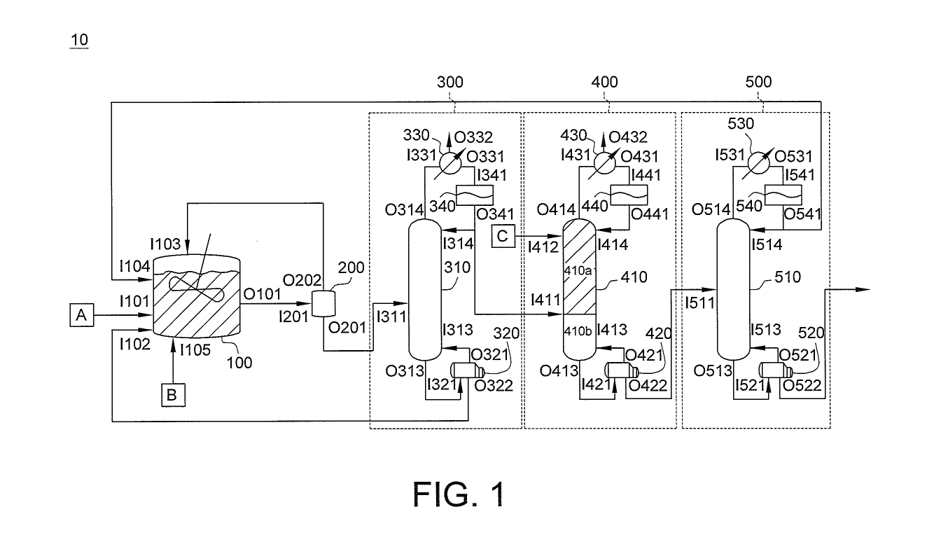

FIG. 1 is schematic diagram showing formic acid manufacturing system according to a first embodiment of the invention. Formic acid manufacturing system 10 includes a reactor 100, a first separation device 200, a second separation device 300, a reactive distillation device 400 and a third separation device 500. The reactor 100 can be a continuous stirred-tank reactor (CSTR) or a plug flow reactor (PFR). The first separation device 200 includes a flash column. The second separation device 300 and the third separation device 500 include a distillation column respectively. The reactive distillation device 400 includes a reactive distillation column.

The following contents further describe the devices of formic acid manufacturing system 10. The pressure of the reactor 100 is between 20 and 50 atm and the temperature of the reactor 100 is between 60° C. and 100° C. The carbon monoxide and the methanol are conveyed to the reactor 100. The carbon monoxide and the methanol are provided by a carbon monoxide loading chamber A and a methanol loading chamber B respectively. The carbon monoxide and the methanol are fed into the reactor 100 from the inlet I101 and I105 respectively. The chemical formula (III) shows that the net reaction does not include methanol, thus the makeup of methanol is to supply the methanol which is loss from the formic acid manufacturing system 10. Methyl formate is produced by processing a carbonylation reaction of carbon monoxide and methanol in the presence of a heterogeneous catalyst. The chemical formula (I) is

CH3OH+CO [symbol] HCOOCH3 (I)

The heterogeneous catalyst is, for example, sodium methoxdie or ion-exchange resin. The first mixture which is output from the outlet O101 of the reactor 100 is produced by the reaction of carbon monoxide and methanol in the reactor 100. The first mixture mainly includes carbon monoxide, methanol and methyl formate.

In this embodiment, the first separation device 200 can include a flash column which is used for vapor-liquid separation. The pressure of the first separation device 200 is between 3 and 5 atm and the temperature of the first separation device 200 is between 30° C. and 70° C. The first mixture is conveyed to the first separation device 200 from the inlet I201 of the first separation device 200. The first mixture separates into the second mixture and the third mixture by the first separation device 200. The second mixture is output from the outlet O202 of the first separation device 200 and input to the inlet I103 of the reactor 100. The second mixture includes plenty of carbon monoxide and slight amount of methanol and methyl formate. The second mixture has a lower boiling point and is substantially gas phase. Because the second mixture is rich in carbon monoxide which is the reactant of conbonylation reaction occurred in the reactor 100, recycle the second mixture to the reactor 100 can help the conbonylation reaction process toward production. Due to most carbon monoxide being recycled to the reactor 100, the main components of the third mixture are methanol and methyl formate.

In this embodiment, the second separation device 300 can include distillation column. The second separation device 300 is used for recycling plenty of methanol in the third mixture to the reactor 100 to facilitate the conbonylation reaction processing toward production. The pressure of the second separation device 300 is between 3 and 5 atm. The second separation device 300 includes the distillation column 310, the reboiler device 320, the condenser device 330 and the reflux device 340. In the bottom part of the second separation device 300, a mixture is output from the outlet O313 of the distillation column 310 and input to the inlet I321 of the reboiler device 320. A mixture is output from the outlet O321 of the reboiler device 320 and input to the inlet I313 of the distillation column 310. The fourth mixture which mainly includes methanol which has higher boiling point in the third mixture is output from the outlet O322 of the reboiler device 320. The fourth mixture which is rich in methanol is input to the inlet I102 of the reactor 100.

On the top part of the second separation device 300, the inlet I331 of the condenser device 330 receives a mixture which is output from the outlet O314 of the distillation column 310. The inlet I341 of the reflux device 340 receives a mixture which is output from the outlet O331 of the condenser device 330. The fifth mixture which mainly includes the methanol and methyl formate of the third mixture is output from the outlet O341 of the reflux device 340. Part of the fifth mixture is input to the inlet I314 of the distillation column 310 while the other part of the fifth mixture is input to the reactive distillation device 400. Besides, there is still a little of carbon monoxide dissolved in the third mixture. The carbon monoxide in the third mixture may be vaporized to cause the pressure of the second separation device 300 increase because of the temperature of the second separation device 300. The outlet O332 can be designed on the condenser device 330 for discharging the carbon monoxide which is vaporized in the second separation device 300 to prevent the pressure of the second separation device 300 too high for security concerns.

In this embodiment, the reactive distillation device 400 can include reactive distillation column. The reactive distillation device 400 is used for producing formic acid and methanol by hydrolyzing methyl formate of the fifth mixture with an external water. The pressure of the reactive distillation device 400 is between 3 and 5 atm. The reactive distillation device 400 includes the reactive distillation column 410, the reboiler device 420, the condenser device 430 and the reflux device 440. The reactive distillation column 410 includes several reactive trays and stripping trays (not shown in FIGS). Each reactive tray is filled with the heterogeneous catalyst for the mixture on the reactive tray processing a reaction. The heterogeneous catalyst on the reactive trays can be ion-exchange resin. The stripping trays which are located below the reactive trays are used for separating the mixture on the stripping trays. In an embodiment, the number of the reactive trays is between 14 and 24 and the number of the stripping trays is between 11 and 21. The number of these trays is related to the flow rate of the external carbon monoxide and water which are conveyed to the formic acid manufacturing system 10. The region which has reactive trays in the reactive distillation column 410 is defined as the reactive zone 410a, while the region which has stripping trays in the reactive distillation column 410 is defined as the stripping zone 410b. The reaction occurred on the reactive trays is the hydrolysis reaction. The reactants of the hydrolysis reaction are methyl formate and water. The productions of the hydrolysis reaction are formic acid and methanol. The hydrolysis reaction can be shown as chemical formula (II):

HCOOCH3+H2O [symbol] HCOOH+CH3OH (II)

The fifth mixture which is output from the second separation device 300 and input to the inlet I411 of the reactive distillation column 410 mainly includes methanol and methyl formate. An external water which is input to the inlet I412 of the reactive distillation column 410 is provided by the water loading chamber C. The external water is conveyed to at least one of the reactive trays. The position of the inlet I412 in the reactive distillation column 410 is located higher than the position of the inlet I411 in the reactive distillation column 410 because the boiling point of the water is higher than the fifth mixture. In other words, the position of the tray which is fed by the fifth mixture is located lower than the position of the tray which is fed by the water. The position of the tray which is fed by the water is the higher the better. Preferably, the external water is conveyed to the highest reactive tray. After the water which has higher boiling point in the reactants of the hydrolysis reaction is input to the higher tray in the reactive distillation column 410, the water can move to the bottom of the reactive distillation column 410. In addition, after the methyl formate which has lower boiling point in the reactants of the hydrolysis reaction is input to the lower tray in the reactive distillation column 410, the methyl formate can be vaporized to move to the top of the reactive distillation column 410. In this way, the most amount of water and methyl formate process a reaction to produce formic acid and methanol in the reactive zone 410a.

On the top part of the reactive distillation device 400, the inlet I431 of the condenser device 430 receives a mixture which is output from the outlet O414 of the reactive distillation column 410. The inlet I441 of the reflux device 440 receives a mixture which is output from the outlet O431 of the condenser device 430. A mixture which is output from the outlet O441 of the reflux device 440 is input to the inlet I414 of the reactive distillation column 410. By adopting total reflux in the condenser device 430 and reflux device 440, the unreacted methyl formate can be maintained on the top of the reactive distillation device 400. The reflux device 440 also can be filled with the heterogeneous catalyst. In this way, most methyl formate be kept on the top of the reactive distillation device 400 to make the reaction conversion maximum and produce more formic acid.

In the bottom part of the reactive distillation device 400, a mixture is output from the outlet O413 of the reactive distillation column 410 and is input to the inlet I421 of the reboiler device 420. A mixture is output from the outlet O421 of the reboiler device 420 and input to the inlet I413 of the reactive distillation column 410. The sixth mixture which mainly includes unreacted water and part of formic acid and methanol is output from the outlet O422 of the reboiler device 420. The amount of methyl formate in the sixth mixture has to be controlled at the minimum level, because the methyl formate may recycle to the reactor 100. After the unreacted methyl formate is conveyed to the third separation device 400 (described later), the unreacted methyl formate may recycle to the reactor 100 to lower the conversion of the carbonylation reaction occurred in the reactor 100. Besides, there is still a little of carbon monoxide dissolved in the fifth mixture. The carbon monoxide in the fifth mixture may be vaporized to cause the pressure of the reactive distillation device 400 increase because of the temperature of the reactive distillation device 400. The outlet O432 can be designed on the condenser device 430 for discharging the carbon monoxide vaporized in the reactive distillation 400 to prevent the pressure of the reactive distillation device 400 too high for security concerns.

In this embodiment, the third separation device 500 can include distillation column. The third separation device 500 is used for recycling plenty of methanol of the sixth mixture to the reactor 100 to facilitate the conbonylation reaction processing toward production. The pressure of the third separation device 500 is between 3 and 5 atm. The third separation device 500 includes the distillation column 510, the reboiler device 520, the condenser device 530 and the reflux device 540. On the top part of the third separation device 500, the inlet I531 of the condenser device 530 receives a mixture which is output from the outlet O514 of the distillation column 510. The inlet I541 of the reflux device 540 receives a mixture which is output from the outlet O531 of the condenser device 530. The seventh mixture which mainly includes the methanol of the sixth mixture is output from the outlet O541 of the reflux device 540. Part of the seventh mixture is input to the inlet I514 of the distillation column 510 while the other part of the seventh mixture is input to the reactor 100. A pump can be disposed between the reactor 100 and the third separation device 500 to make the seventh mixture be conveyed to the reactor 100, because the pressure of the reactor 100 is higher than the pressure of the third separation device 500.

In the bottom part of the third separation device 500, a mixture is output from the outlet O513 of the distillation column 510 and is input to the inlet I521 of the reboiler device 520. A mixture is output from the outlet O521 of the reboiler device 520 and is input to the inlet I513 of the distillation column 510. The eighth mixture which mainly includes formic acid and water which has higher boiling point in the sixth mixture is output from the outlet O522 of the reboiler device 520. In this embodiment, for example, the concentration of formic acid of the eighth mixture can meet 85 weight percent.

In the view of the total formic acid manufacturing system 10, the net reaction is that the water reacts with the carbon monoxide to produce the formic acid as shown in the chemical formula (III). In order to produce high purity formic acid, the mole ratio between the external water to the external carbon monoxide which are conveyed to the formic acid manufacturing system 10 substantially is 1 to 1 during a constant time period. In this way, it can prevent unreacted reactants be blended with the production formic acid.

H2O+CO [symbol] HCOOH (III)

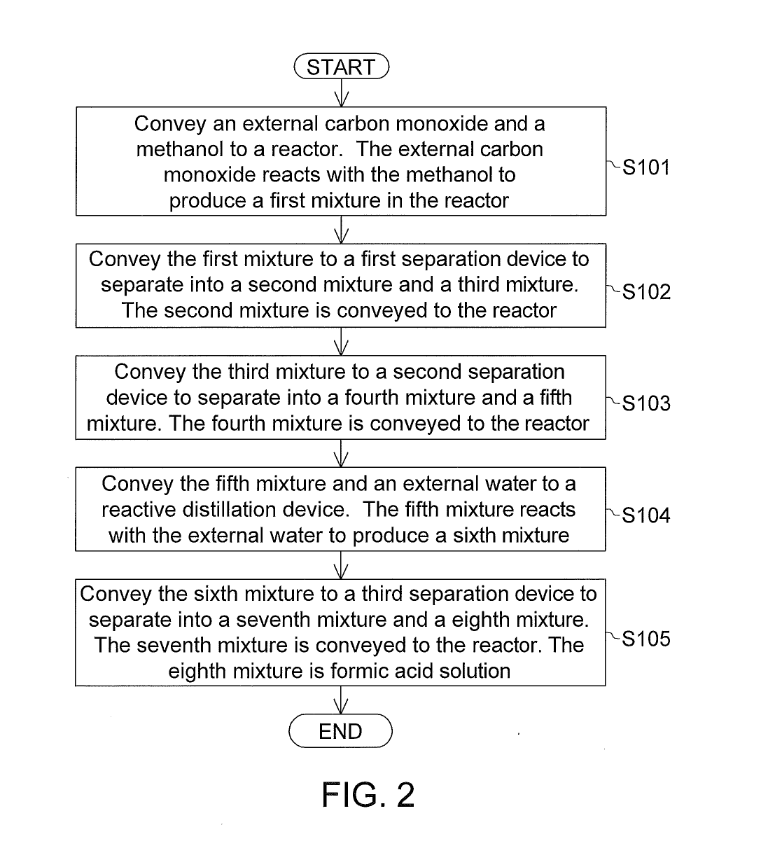

Each device described above with a flow chart to describe the operation of each device. However, the flow chart is not limited to use on the elements and devices described above. FIG. 2 shows a flow chart showing the method of manufacturing formic acid according to a first embodiment of the invention. First, in the step S101, convey an external carbon monoxide and a methanol to a reactor. The external carbon monoxide reacts with the methanol to produce a first mixture in the reactor. Then, in the step S102, convey the first mixture to a first separation device to separate into a second mixture and a third mixture. The second mixture is conveyed to the reactor. And then, in the step S103, convey the third mixture to a second separation device to separate into a fourth mixture and a fifth mixture. The fourth mixture is conveyed to the reactor. Then, in the step S104, convey the fifth mixture and an external water to a reactive distillation device. The fifth mixture reacts with the external water to produce a sixth mixture. And then, in the step S105, convey the sixth mixture to a third separation device to separate into a seventh mixture and a eighth mixture. The seventh mixture is conveyed to the reactor. The eighth mixture is formic acid solution.

The following is a preferred application. The reactor is a CSTR. The first separation device is a flash column. The second and third separation devices are distillation columns. The reactive distillation device is reactive distillation column device. The number of the tray is counted from top.

| Item | Application |
|---|---|
| CSTR and the first separation device | |
| Cabon monoxide flowrate (kmole/hr) | 100 |
| Methanol flowrate (kmole/hr) | 1.3 |
| CSTR residual time (min) | 10 |
| CSTR volumn (I) | 4084 |
| CSTR pressure (atm) | 40 |
| CSTR temperature (° C.) | 80 |
| Flash residual time (min) | 10 |
| Flash volumn (I) | 4084 |
| Flash pressure (atm) | 4 |
| Flash temperature (° C.) | 50 |
| The second separation device | |
| Total No. of trays | 10 |
| The third mixture feed tray | 3rd |
| Distillation column pressure (atm) | 4 |
| Distillation column diameter (m) | 0.99 |
| Reactive distillation device | |
| Total No. of trays | 35 |
| Number of reactive trays | 19 |
| Number of stripping trays | 16 |
| Position of reactive trays | 1st~19th |
| Water feed tray | 1st |
| The fifth mixture feed tray | 33th |
| Catalyst amount in each reactive tray/total | 0.15/2.85 |
| catalyst amount (m3) | |
| Water flow rate (kmole/hr) | 100 |
| Reactive distillation column pressure (atm) | 4 |
| Reactive distillation column diameter (m) | 1.92 |
| The third separation device | |
| Total No. of trays | 13 |
| The third mixture feed tray | 8th |
| Distillation column pressure (atm) | 4 |
| Distillation column diameter (m) | 1.12 |
| Formic acid purity of the eighth mixture | 85 |
| (weight percent) | |

The reactive distillation device 400 is applied to formic acid manufacturing system 10 to make separation and reaction process occurred at the same device in this embodiment. Compared to make the separation and reaction process occurred at different unit, this embodiment can save more manufacturing cost and produce high purity formic acid.

### Second Embodiment

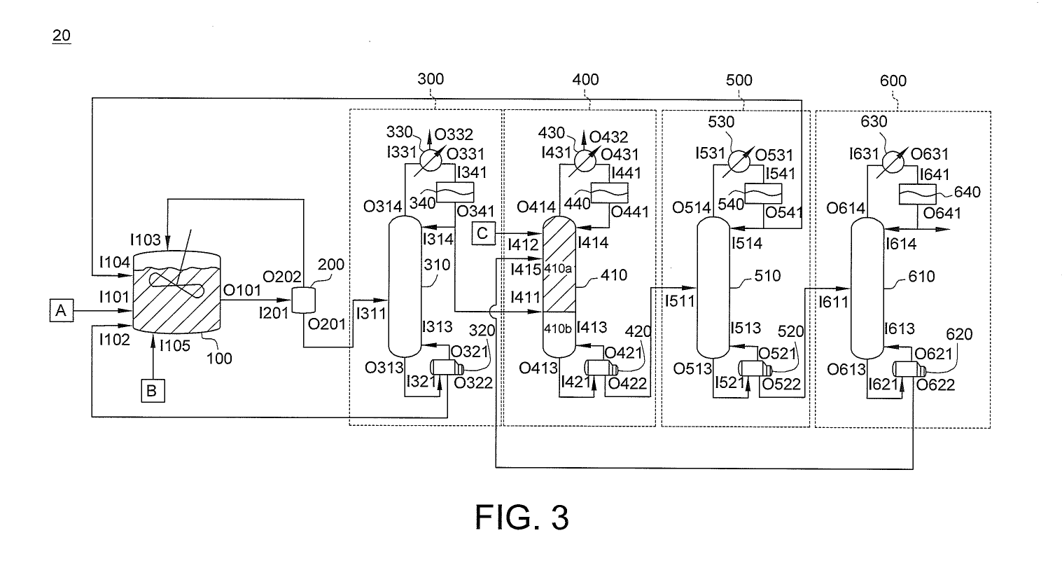

FIG. 3 is schematic diagram showing formic acid manufacturing system according to a second embodiment of the invention. In the second embodiment, the formic acid manufacturing system 20 further includes a fourth separation device 600. The reactor 100, the first separation device 200, the second separation device 300, the reactive distillation device 400, the third separation device 500, input location, output location and reaction are similar with the first embodiment, therefore the similar parts will not be described again.

The fourth separation device 600 can include a distillation column. The fourth separation device 600 is used for recycling part of formic acid and water of the eighth mixture to the reactive distillation device 400 to facilitate the hydrolysis reaction processing toward production. The pressure of the fourth separation device 600 is between 3 and 5 atm. The fourth separation device 600 includes the distillation column 610, the reboiler device 620, the condenser device 630 and the reflux device 640. In the bottom part of the fourth separation device 600, a mixture is output from the outlet O613 of the distillation column 610 and is input to the inlet I621 of the reboiler device 620. A mixture is output from the outlet O621 of the reboiler device 620 and is input to the inlet I613 of the distillation column 610. The ninth mixture which mainly includes formic acid and water is output from the outlet O622 of the reboiler device 620. The composition of formic acid and water which is output from the outlet O622 approach their azeotropic composition. The ninth mixture is conveyed to at least one of the reactive trays and is input to the inlet I415 of the reactive distillation column 410. The position of the inlet 1415 in the reactive distillation column 410 is located higher than the position of the inlet I411 in the reactive distillation column 410.

On the top part of the third separation device 600, the inlet I631 of the condenser device 630 receives a mixture which is output from the outlet O614 of the distillation column 610. The inlet I641 of the reflux device 640 receives a mixture which is output from the outlet O631 of the condenser device 630. The tenth mixture which mainly includes the high purity formic acid is output from the outlet O641 of the reflux device 640. Part of the tenth mixture is input to the inlet I614 of the distillation column 610 while the other part of the tenth mixture is output to be production. In this embodiment, the water in the third separation device 600 can be recycled to the reactive distillation device 400 to hydrolyze with the methyl formate to raise the conversion of hydrolysis reaction and consume the water. In this way, there only have a little water in the tenth mixture. Thus, the tenth mixture can be high purity formic acid solution. For example, the concentration of the formic acid of the eighth mixture can meet 99 weight percent.

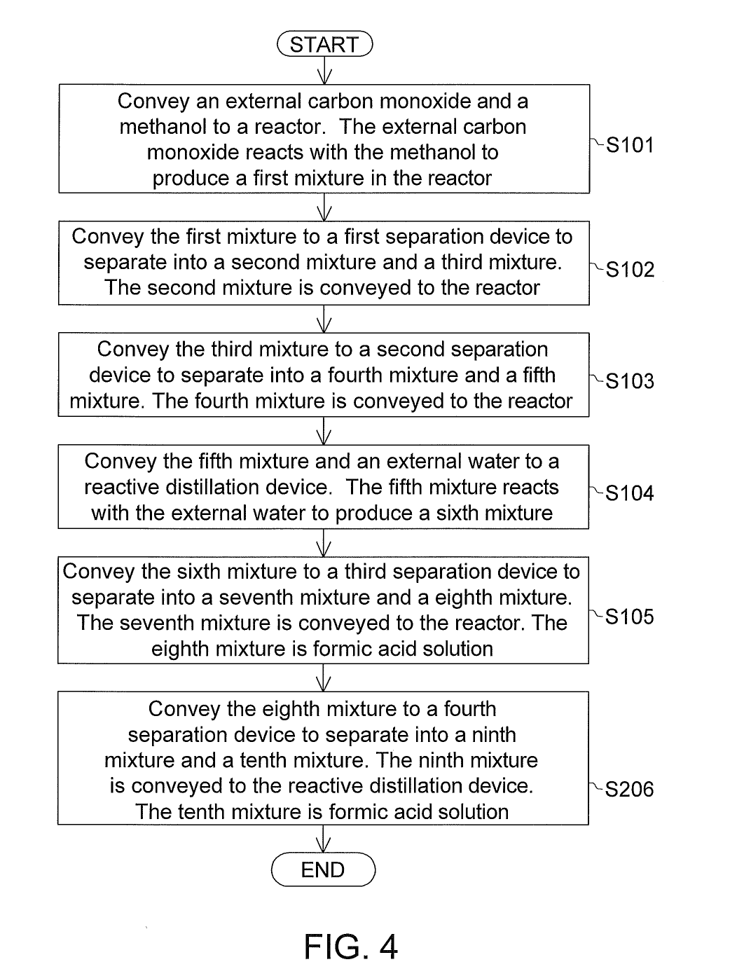

Each device described above with a flow chart to describe the operation of each device. However, the flow chart is not limited to use on the elements and devices described above. FIG. 4 shows a flow chart showing the method of manufacturing formic acid according to a second embodiment of the invention. In the step S206, convey the eighth mixture to a fourth separation device to separate into a ninth mixture and a tenth mixture. The ninth mixture is conveyed to the reactive distillation device. The tenth mixture is formic acid solution. The following is a preferred application. The fourth separation device is a distillation column.

| Item | Application |
|---|---|
| CSTR and the first separation device | |
| Cabon monoxide flowrate (kmole/hr) | 100 |
| Methanol flowrate (kmole/hr) | 1.3 |
| CSTR residual time (min) | 10 |
| CSTR volumn (I) | 4786 |
| CSTR pressure (atm) | 40 |
| CSTR temperature (° C.) | 80 |
| Flash residual time (min) | 10 |
| Flash volumn (I) | 4786 |
| Flash pressure (atm) | 4 |
| Flash temperature (° C.) | 50 |
| The second separation device | |
| Total No. of trays | 10 |
| The third mixture feed tray | 3rd |
| Distillation column pressure (atm) | 4 |
| Distillation column diameter (m) | 1.16 |
| Reactive distillation device | |
| Total No. of trays | 38 |
| Number of reactive trays | 22 |
| Number of stripping trays | 16 |
| Position of reactive trays | 1st~22th |
| Water feed tray | 1st |
| The fifth mixture feed tray | 33th |
| The ninth mixture feed tray | 1st |
| Catalyst amount in each reactive tray/total | 0.15/3.3 |
| catalyst amount (m3) | |
| Water flow rate (kmole/hr) | 100 |
| Reactive distillation column pressure (atm) | 4 |
| Reactive distillation column diameter (m) | 2.76 |
| The third separation device | |
| Total No. of trays | 33 |
| The third mixture feed tray | 28th |
| Distillation column pressure (atm) | 4 |
| Distillation column diameter (m) | 1.51 |
| The fourth separation device | |
| Total No. of trays | 15 |
| The third mixture feed tray | 10th |
| Distillation column pressure (atm) | 4 |
| Distillation column diameter (m) | 0.90 |
| Formic acid purity of the tenth mixture | 99 |
| (weight percent) | |

The reactive distillation device applied in the formic acid manufacturing system can combine separation process and reaction process into one device in above embodiments. In this way, the formic acid manufacturing system can not only save the space in the factory, but also save the cost of the factory. Besides, the formic acid produced by these embodiments has high purity and high economic value. The formic acid manufacturing system can save cost and increase the value of the product at the same. The embodiments have great economic value in the industry field.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A formic acid manufacturing system, comprising:
a reactor, an external carbon monoxide reacting with a methanol to produce a first mixture in the reactor;
a first separation device for receiving the first mixture, the first separation device separating the first mixture into a second mixture and a third mixture, the second mixture being conveyed to the reactor;
a second separation device for receiving the third mixture, the second separation device separating the third mixture into a fourth mixture and a fifth mixture, the fourth mixture being conveyed to the reactor;
a reactive distillation device for receiving the fifth mixture and an external water, the fifth mixture reacting with the external water to produce a sixth mixture in the reactive distillation device; and
a third separation device for receiving the sixth mixture, the third separation device separating the sixth mixture into a seventh mixture and a eighth mixture, the seventh mixture being conveyed to the reactor, the eighth mixture being formic acid solution.

2. The formic acid manufacturing system according to claim 1, wherein the reactive distillation device comprises a reactive distillation column, the reactive distillation column comprises:
a plurality of reactive trays, each of the reactive trays being filled with a heterogeneous catalyst; and
a plurality of stripping trays located below the reactive trays.

3. The formic acid manufacturing system according to claim 2, wherein the number of the reactive trays is between 14 and 24.

4. The formic acid manufacturing system according to claim 2, wherein the number of the stripping trays is between 11 and 21.

5. The formic acid manufacturing system according to claim 2, wherein the external water is conveyed to at least one of the reactive trays.

6. The formic acid manufacturing system according to claim 2, wherein the external water is conveyed to the highest reactive trays.

7. The formic acid manufacturing system according to claim 1, further comprising:
a fourth separation device for receiving the eighth mixture, the fourth separation device separating the eighth mixture into a ninth mixture and a tenth mixture, the tenth mixture being formic acid solution which is purer than the formic acid solution in the eighth solution.

8. The formic acid manufacturing system according to claim 7, wherein the reactive distillation device comprises a plurality of trays, the position of the tray which the ninth mixture is conveyed to in the reactive distillation device being located lower than the position of the tray which the external water is conveyed to in the reactive distillation device.

9. The formic acid manufacturing system according to claim 7, wherein the fourth separation device comprises a distillation column.

10. The formic acid manufacturing system according to claim 7, wherein the main components of the ninth mixture are formic acid and water.

11. The formic acid manufacturing system according to claim 1, wherein the first separation device comprises a flash column which makes the first mixture liquid-vapor separated.

12. The formic acid manufacturing system according to claim 1, wherein the reactive distillation device comprises a plurality of trays, the position of the tray which the fifth mixture is conveyed to in the reactive distillation device being located lower than the position of the tray which the external water is conveyed to in the reactive distillation device.

13. The formic acid manufacturing system according to claim 1, wherein the mole ratio of the external water to the external carbon monoxide substantially is 1 to 1.

14. The formic acid manufacturing system according to claim 1, wherein the main component of the fourth mixture is methanol.

15. The formic acid manufacturing system according to claim 1, wherein the main component of the seventh mixture is methanol.

16. The formic acid manufacturing system according to claim 1, wherein the second separation device and the third separation device comprise a distillation column respectively.

17. The formic acid manufacturing system according to claim 1, wherein the reactive distillation device is a total reflux reactive distillation device.

18. A method of manufacturing formic acid, comprising:
conveying an external carbon monoxide and a methanol to a reactor, the external carbon monoxide reacting with the methanol to produce a first mixture in the reactor;
conveying the first mixture to a first separation device to separate into a second mixture and a third mixture, the second mixture being conveyed to the reactor;
conveying the third mixture to a second separation device to separate into a fourth mixture and a fifth mixture, the fourth mixture being conveyed to the reactor;
conveying the fifth mixture and an external water to a reactive distillation device, the fifth mixture reacting with the external water to produce a sixth mixture; and
conveying the sixth mixture to a third separation device to separate into a seventh mixture and a eighth mixture, the seventh mixture being conveyed to the reactor, the eighth mixture being formic acid solution.

19. The method according to claim 18, further comprising conveying the eighth mixture to a fourth separation device to separate into a ninth mixture and a tenth mixture, the ninth mixture being conveyed to the reactive distillation device, the tenth mixture being formic acid solution.
